(19) United States
(12) Patent Application Publication
(10) Pub. No.: US 2004/0230314 A1
(43) Pub. Date: Nov. 18, 2004

(54) ENVIRONMENTALLY-FRIENDLY CADAVERIC DONOR PROSTHESES

(76) Inventor: Richard Lee Marrell, Valparaiso, IN (US)

Correspondence Address: HARTMAN & HARTMAN, P.C., 552 EAST 700 NORTH, VALPARAISO, IN, 46383, US

(21) Appl. No.: 10/709,535

(22) Filed: May 12, 2004

Related U.S. Application Data
(60) Provisional application No. 60/469,690, filed on May 12, 2003.

(51) Int. Cl. A61F 2/28

(52) U.S. Cl. 623/23.47; 623/23.58; 27/21.1

(57) ABSTRACT

Prosthetic devices suitable for replacing bone material that has been extracted from a cadaver, such as for use as transplants. The prosthetic devices include multiple elongate members and devices for securing the elongate members together. According to one embodiment, a first elongate member has a longitudinal axis, oppositely-disposed first and second ends, an exterior surface between the first and second ends, and a channel in the exterior surface and contiguous with the second end. A second elongate member is disposed in the channel so as to have a first end disposed in the channel and a second end that extends beyond the second end of the first elongate member. The elongate members and the securing device are formed from materials that do not release carcinogenic byproducts when burned, such as wood or another cellulosic material.

### CROSS REFERENCE TO RELATED APPLICATIONS

This application claims the benefit of U.S. Provisional Application No. 60/469,690, filed May 12, 2003.

### BACKGROUND OF INVENTION

1. Field of the Invention

The present invention generally relates to prosthetic devices suitable for replacing bone material that has been extracted from cadavers, such as for use as transplants.

More particularly, this invention relates to prosthetic devices formed of materials that avoid the need for explantation of the devices in cases where the cadaver is to be cremated.

2. Description of the Related Art

The rate of organ and tissue donorship to tissue banks is growing each year. One example is the transplantation of bone material for the purpose of treating bone diseases and abnormalities. Removal of bone material from a body can result in unacceptable disfiguration of the body. As such, osteoprosthetic devices have been proposed, such as those disclosed in U.S. Pat. Nos. 4,863,473, 5,496,373, and 5,683,462. These patents generally disclose devices comprising tubular telescoping members that permit the devices to be adjusted in length for the particular size of the bone being replaced. Plastic tubing is said to be preferred, such as polyvinylchloride (PVC) tubing typically used for household plumbing. U.S. Pat. No. 4,863,473 also mentions metal and wood as alternative materials for the telescoping members.

Cremation is also currently growing in popularity. Because of this trend, tissue banks are increasingly having difficulty with the existing plastic prosthetic devices, particularly those made from PVC. Specifically, PVC is not crematory compatible because byproducts of its combustion are carcinogenic and can damage a crematorium retort. Therefore, PVC prosthetics should be explanted before a body is cremation. However, prosthetic implantation may be in question where families of the deceased have not decided on funeral arrangements and direct cremation from a hospital is a possibility. If tissue bank communications with postmortem caretakers break down, explantation of the PVC prosthetics may not occur before cremation. Another drawback associated with the need to explant PVC prosthetics is that explantation creates additional work for funeral homes, and can lead to additional expenses to the tissue bank in the form of reimbursements to funeral homes that charge the cost of explantation back to the tissue bank.

In view of the above, it would be desirable if osteoprosthetic devices were available that are simpler to use and implant and were more environmentally friendly to eliminate the need for their removal before cremation.

### SUMMARY OF INVENTION

The present invention provides prosthetic devices suitable for replacing bone material that has been extracted from a cadaver, such as for use as transplants. The prosthetic devices of this invention are formed of materials that avoid the need for explantation of the devices in cases where the cadaver is to be cremated.

According to one aspect of the invention, a prosthetic device comprises first and second elongate members and means for securing the elongate members together. The first elongate member has a longitudinal axis, oppositely-disposed first and second ends, an exterior surface between the first and second ends, and a channel in the exterior surface and contiguous with the second end. The second elongate member is disposed in the channel so as to have a first end disposed in the channel and a second end that extends beyond the second end of the first elongate member. The securing means is operable to secure the first and second elongate members together while the second elongate member is disposed in the channel. The first and second elongate members and the securing means are formed from materials that do not release carcinogenic byproducts when burned. Wood is a preferred material for the prosthetic device, though the use of other cellulosic materials is foreseeable.

According to a second aspect of the invention, a prosthetic device comprises multiple elongate members of different lengths, and means for securing the elongate members together. The elongate members are longitudinally aligned in series with each other, with their adjacent ends facing but spaced apart from each other, and being secured together with the securing means. The elongate members are again formed from materials that do not release carcinogenic byproducts when burned.

Other objects and advantages of this invention will be better appreciated from the following detailed description.

### BRIEF DESCRIPTION OF DRAWINGS

FIG. 1 shows a human skeleton and indicates the placement of three prosthetic devices in accordance with embodiments of this invention.

FIGS. 2, 3 and 4 are more detailed views of the prosthetic devices shown in FIG. 1.

FIGS. 5, 6 and 7 show additional prosthetic devices in accordance with embodiments of this invention.

### DETAILED DESCRIPTION

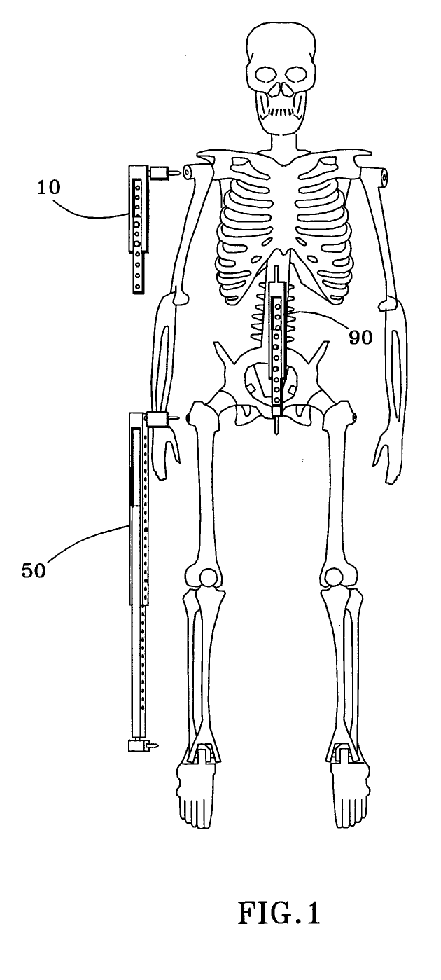

Three prosthetic devices 10, 50 and 90 are illustrated in FIG. 1 in accordance with embodiments of this invention. The locations in the human body where the devices 10, 50 and 90 would be implanted, i.e., where the devices 10, 50 and 90 are adapted for implanting as prostheses, are indicated in FIG. 1 as being the humerus, leg bones, and bones in the spinal column, respectively.

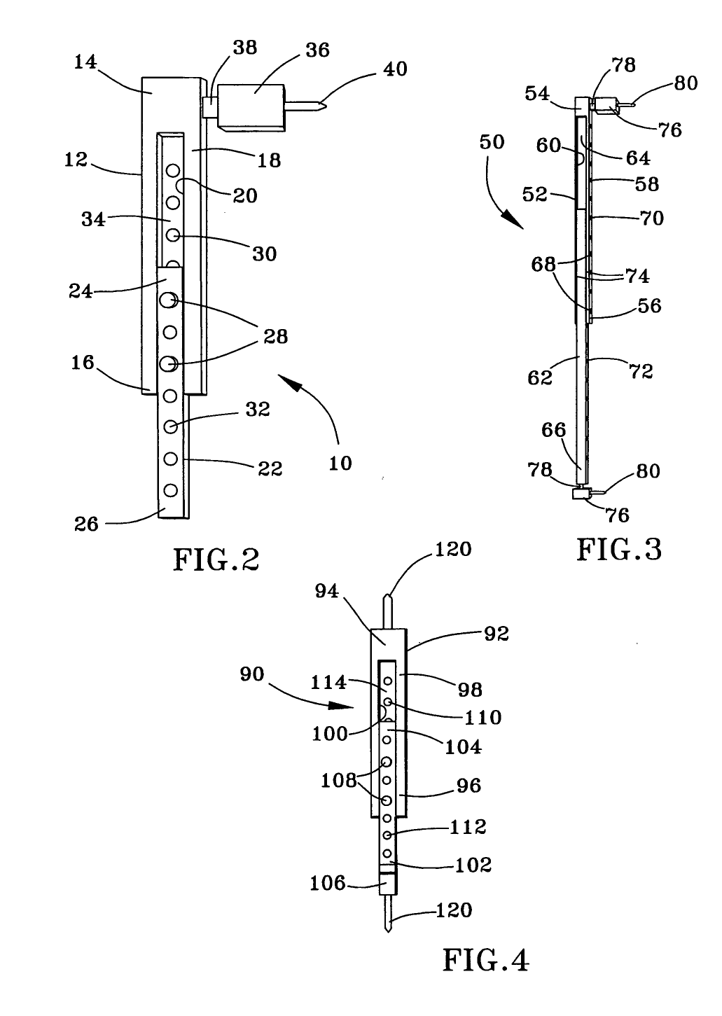

Each of the devices 10, 50 and 90 generally comprises a pair of elongate members that are secured together in a manner that enables the lengths of the devices 10, 50 and 90 to be adjusted. With specific reference to the device 10, shown in greater detail in FIG. 2 (not to scale relative to FIGS. 3 and 4), the device 10 has a first elongate member 12 with oppositely-disposed ends 14 and 16, an exterior surface 18, and a channel 20 in the exterior surface 18. The channel 20 is shown as being contiguous with the lower end 16 of the member 12, but not the upper end 14 of the member 12. A second elongate member 22 is disposed in the channel 20 so as to have one end 24 disposed in the channel 20 while its opposite end 26 extends beyond the lower end 16 of the first elongate member 12. The elongate members 12 and 22 are shown as being secured together with two pins 28 received in complementary bores 30 and 32 formed in the elongate members 12 and 22, respectively. The bores 30 in the first elongate member 12 are defined in a base wall 34 of the channel 20, such that the pins 28 are oriented in a direction normal to the surface 18 of the member 12.

The elongate member 12 and the channel 20 are each depicted as having a substantially rectilinear cross-section, with at least that portion of the elongate member 22 received in the channel 20 having a cross-sectional shape complementary to that of the channel 20 so that the elongate member 22 is securely nested in the channel 20. Suitable cross-sectional dimensions for the member 12 and the channel 20 are about ¾×1.5 inches and about ⅝×1 inch, respectively. The member 22 can have cross-sectional dimensions of about 1×1 inch. That portion of the member 22 protruding outside the channel 20 can have a more rounded shape. The members 12 and 22 can have any suitable length, and the ability to selectively align and pin pairs of the bores 30 and 32 enables the placement of the member 22 within the channel 20 to be adjusted so that the device 10 can achieve a range of useful lengths.

FIG. 2 further shows the device 10 as including a fixation block 36 intended for placement in the glenoid fossa of the cadaver. The block 36 is secured with a dowel pin 38 to one side of the elongate member 12 near its upper end 14, such that the block 36 extends from the member 12 in a direction transverse to the longitudinal axis of the member 12. An optional threaded fastener 40 is shown as extending from the block 36 also in the transverse direction to enable the device 10 to be secured to, for example, the glenoid fossa or other suitable anchor. If so desired, a second fixation block (not shown) can be secured to the lower end 26 of the second elongate member 22 for anchoring the device 10 in the elbow region of the body, or to secure a second prosthetic device (not shown). While the ends 14, 16, 24 and 26 of the members 12 and 22 are shown as being square, it is foreseeable that, for example, the corner of the member 12 opposite the block 36 could be beveled for aesthetic contouring in the shoulder/deltoid region of the body.

According to a preferred aspect of the invention, the elongate members 12 and 22, pins 28 and block 36 are each formed from materials that do not release carcinogenic byproducts when burned. From the standpoint of cost and availability, wood is a preferred material for the prosthetic device 10, though the use of other cellulosic materials is foreseeable.

With reference to FIG. 3, the device 50 is shown as being adapted to replace the femur, tibia and fibula. As with the device 10 of FIG. 2, the device 50 comprises a pair of elongate members 52 and 62, with the member 62 disposed in a channel 60 defined in a surface 58 of the member 52 so that an upper end 64 of the member 62 is disposed in the channel 60 while its lower end 66 extends beyond the lower end 56 of the member 52. The members 52 and 62 are secured together with two pins 68 received in complementary bores 70 and 72 formed in the members 52 and 62, respectively. The bores 70 in the first elongate member 52 are defined in a pair of sidewalls 74 defined by the channel 60, and the pins 68 engage the member 62 from the side. As with the previous embodiment, the elongate members 52 and 62 and the channel 60 are each depicted as having substantially rectilinear cross-sections, with a portion of the elongate member 62 being complementary to the channel 60 so that the member 62 is securely nested in the channel 60.

FIG. 3 further shows the device 50 as including fixation blocks 76 secured near the upper end 54 of the member 52 and at the lower end 66 of the member 62. The blocks 76 are shown as being secured with dowel pins 78 to their respective ends 54 and 66, with threaded fasteners 80 extending from the blocks 76 in the same transverse direction to the longitudinal axis of the first elongate member 52. The locations of the pins 78 on the members 52 and 62 establish whether the device 50 is configured for use on the righthand or lefthand side of the body. The upper block 76 enables the device 50 be secured to the hip bone, while the lower block 76 enables the device 50 to be secured to the ankle. As with the previous embodiment, the ends 54, 56, 64 and 66 of the members 52 and 62 can be square as shown, or beveled. For example, the corner of the member 62 opposite its block 76 could be beveled for aesthetic contouring purposes.

The device 90 shown in FIG. 4 is adapted to replace several vertebrae of the spinal column. As with the previously described devices 10 and 50, the device 90 comprises a pair of elongate members 92 and 102, with the member 102 disposed in a channel 100 defined in a surface 98 of the member 92 so that an upper end 104 of the member 102 is disposed in the channel 100 while its lower end 106 extends beyond the lower end 96 of the member 92. The members 92 and 102 are secured together with two pins 108 received in two pairs of multiple complementary bores 110 and 112 formed in the elongate members 92 and 102, respectively. The bores 110 in the first elongate member 92 are defined in a base wall 114 of the channel 100. The elongate members 92 and 102 and the channel 100 are again depicted as having substantially rectilinear cross-sections, with a portion of the elongate member 102 being complementary to the channel 100 so that the elongate member 102 is securely nested in the channel 100. The device 90 further includes threaded fasteners 120 extending from the upper end 94 of the first elongate member 92 and from the lower end 106 of the second elongate member 102 in directions parallel to the longitudinal axis of the members 92 and 102.

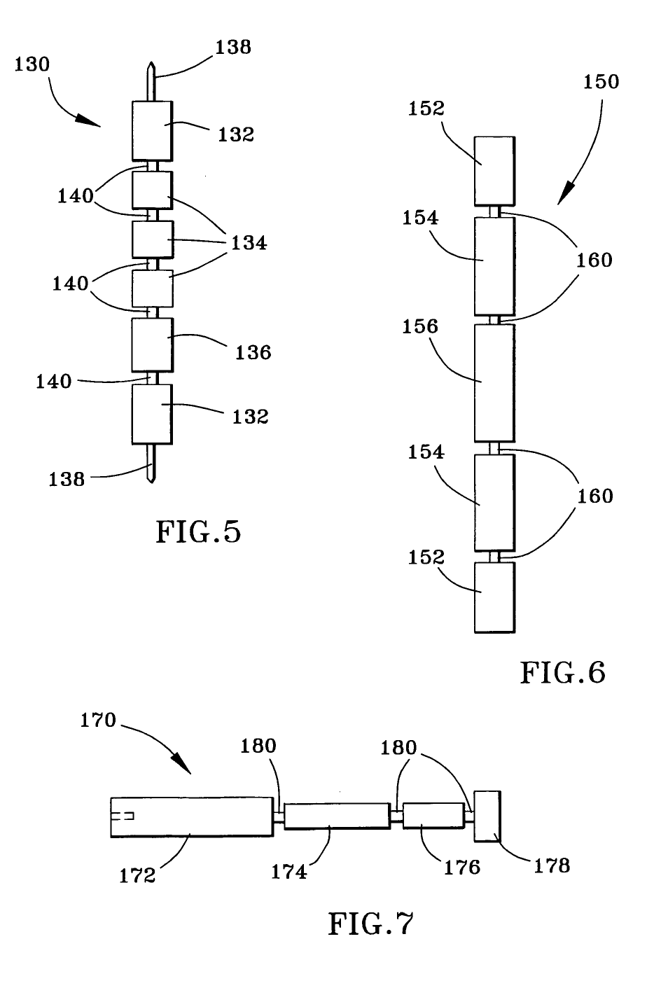

An alternative to the spinal prosthetic device 90 of FIG. 4 is represented in FIG. 5. The device 130 is shown as comprising a number of cylindrically-shaped members 132, 134 and 136 that are coaxially aligned and interconnected to effectively define links of the device 130. The uppermost and lowermost members 132 are equipped with lag screws 138 for fixation to the body and dowels 140 for connection with neighboring members 134 and 136. Each of the intermediate links 134 is equipped with both a dowel 140 and a bore sized to receive the dowel 140 of a neighboring member 132 or 134, thereby enabling interconnection to neighboring members 132 and 134. The member 136 is not equipped with a dowel, but instead is formed to have two bores for engaging dowels 140 of its neighboring members 132 and 134. A preferred diameter for the members 132, 134 and 136 is about 1.25 inches, while preferred lengths for the members 132, 134 and 136 are about 2.0, 1.25 and 1.75 inches, respectively. While six members 132, 134 and 136 are depicted, any number of members 132, 134 and 136 can be assembled to yield a device 130 of suitable length.

FIG. 6 represents a transverse pelvic connection device 150 that can be used to reconstruct the pelvic region of a donor body. The pelvic connection device 150 is essentially identical to the spinal prosthetic device 130 of FIG. 5, except the cylindrically-shaped members 152, 154 and 156 are preferably cut to lengths different than that for the device 130, with the interior-most member 156 being the longest and lacking any dowels. Instead, the member 156 has two bores for receiving dowels 160 of neighboring members 154. The members 152 do not require lag screws, as the device 150 is intended to be secured to the leg prosthetic device 50 of FIG. 3 with the fastener 80 provided therewith. A preferred diameter for the members 152, 154 and 156 is about 1.25 inches, while preferred lengths are about 2.5, 3.5 and 4.0 inches, respectively. Again, any number of members 152, 154 and 156 can be assembled to yield a device 150 of suitable length.

Finally, a prosthetic device 170 adapted as a radius/ulna accessory is shown in FIG. 7. The device 170 is configured for attachment to the device 10 of FIG. 2 and has the functionality of flexion/extension and roticulation. The device 170 is made up of cylindrical members 172, 174, 176 and 178 of different lengths, with the longest 172 also preferably being larger in diameter than the others, e.g., 1-inch diameter versus ⅝-inch diameter. The three longer members 172, 174 and 176 are connected in series with dowels 180, while the shortest member 178 is transversely connected with a dowel 180 to the next longer member 176. The length of the shortest member 178 is intended to create stability in the wrist space, and its transverse attachment relative to the other members 172, 174 and 176 is intended to fill the void left by the procured distal radius/ulna bone. Preferred lengths for the members 172, 174, 176 and 178 are about 5.5, 3.0, 2.0 and 1.5 inches, respectively.

While the invention has been described in terms of a preferred embodiment, it is apparent that other forms could be adopted by one skilled in the art. For example, a variety of fasteners could be used in place of the dowel pins and lag screws set forth in the embodiments of the invention. Therefore, the scope of the invention is to be limited only by the following claims.

1. A prosthetic device comprising:
a first elongate member having a longitudinal axis, oppositely-disposed first and second ends, an exterior surface between the first and second ends, and a channel in the exterior surface and contiguous with the second end;
a second elongate member disposed in the channel so as to have a first end disposed in the channel and a second end that extends beyond the second end of the first elongate member; and
means for securing the second elongate member to the first elongate member while the second elongate member is disposed in the channel;
wherein the first and second elongate members and the securing means are formed from materials that do not release carcinogenic byproducts when burned.

2. The prosthetic device according to claim 1, wherein the first and second elongate members are formed of wood.

3. The prosthetic device according to claim 1, wherein the first elongate member has a substantially rectilinear cross-section.

4. The prosthetic device according to claim 1, wherein the channel defines two walls in the exterior surface on either side of the channel, and the securing means engages the second elongate member and at least one of the walls.

5. The prosthetic device according to claim 4, wherein the securing means comprises a plurality of complementary bores in the second elongate member and at least one of the sidewalls of the first elongate member, and at least one pin received in a pair of the plurality of complementary bores.

6. The prosthetic device according to claim 1, wherein the channel defines two sidewalls in the exterior surface on either side of the channel and a base wall between the sidewalls, and the securing means engages the second elongate member and the base wall.

7. The prosthetic device according to claim 6, wherein the securing means comprises a plurality of complementary bores in the second elongate member and the base wall of the first elongate member, and at least one pin received in a pair of the plurality of complementary bores.

8. The prosthetic device according to claim 1, further comprising a block member secured to the first end of the first elongate member, and fastening means extending from the block member in a direction transverse to the longitudinal axis of the first elongate member.

9. The prosthetic device according to claim 1, further comprising a block member secured to the second end of the second elongate member, the block member having a longitudinal axis transverse to the longitudinal axis of the first elongate member.

10. The prosthetic device according to claim 9, further comprising fastening means extending from the block member in a direction parallel to the longitudinal axis thereof.

11. The prosthetic device according to claim 1, further comprising fastening means extending from the first end of the first elongate member in a direction parallel to the longitudinal axis thereof.

12. The prosthetic device according to claim 1, further comprising fastening means extending from the second end of the second elongate member in a direction parallel to the longitudinal axis of the first elongate member.

13. The prosthetic device according to claim 1, wherein the channel is not contiguous with the first end of the first elongate member.

14. The prosthetic device according to claim 1, wherein the channel and the second elongate member have complementary rectilinear cross-sections.

15. A prosthetic device comprising:
multiple solid elongate members of different lengths, the elongate members being longitudinally aligned in series with each other so as to have adjacent facing ends that are spaced apart from each other; and
means between the facing ends of the elongate members for securing the elongate members together;
wherein the elongate members are formed of materials that do not release carcinogenic byproducts when burned.

16. The prosthetic device according to claim 15, wherein the elongate members have solid cylindrical shapes and substantially equal diameters.

17. The prosthetic device according to claim 15, wherein the securing means comprise wooden dowels.

18. The prosthetic device according to claim 15, further comprising threaded fasteners on at least one of the elongate members located at one end of the prosthetic device.

19. The prosthetic device according to claim 15, further comprising a transverse member secured to one of the elongate members located at one end of the prosthetic device, the transverse member having a solid cylindrical shape and being oriented so as to be transverse to the longitudinal alignment of the elongate members.

20. A prosthetic device comprising:
a first elongate member having a longitudinal axis, oppositely-disposed first and second ends, an exterior surface between the first and second ends, and a channel in the exterior surface, the channel having a rectilinear cross-section that defines two sidewalls in the exterior surface on either side of the channel and a base wall between the sidewalls, the channel being contiguous with the second end but not the first end of the first elongate member;
a second elongate member disposed in the channel so as to have a first end disposed in the channel and a second end that extends beyond the second end of the first elongate member, the second elongate member having a rectilinear cross-section complementary to the rectilinear cross-section of the channel; and
means for securing the second elongate member to the first elongate member while the second elongate member is disposed in the channel, the securing means comprising a plurality of complementary bores in the second elongate member and at least one of the base wall and sidewalls of the first elongate member, and at least one pin received in a pair of the plurality of complementary bores;
wherein the first and second elongate members and the securing means are formed of wood.

21. The prosthetic device according to claim 20, further comprising a block member secured to the first end of the first elongate member, and fastening means extending from the block member in a direction transverse to the longitudinal axis of the first elongate member.

22. The prosthetic device according to claim 20, further comprising a block member secured to the second end of the second elongate member, the block member having a longitudinal axis transverse to the longitudinal axis of the first elongate member.

23. The prosthetic device according to claim 22, further comprising fastening means extending from the block member in a direction parallel to the longitudinal axis thereof.

24. The prosthetic device according to claim 20, further comprising fastening means extending from the first end of the first elongate member in a direction parallel to the longitudinal axis thereof.

25. The prosthetic device according to claim 20, further comprising fastening means extending from the second end of the second elongate member in a direction parallel to the longitudinal axis of the first elongate member.
